(19) United States
(12) Patent Application Publication
(10) Pub. No.: US 2022/0203607 A1
(43) Pub. Date: Jun. 30, 2022

(54) MEDICAL DEVICES, USES AND ADDITIVE MANUFACTURE THEREOF

(71) Applicant: POLY-MED, INC., Anderson, SC (US)

(72) Inventors: Michael Scott Taylor, Anderson, SC (US); Brian Gaerke, Travelers Rest, SC (US); Parimal Patel, Central, SC (US); Ryan Borem, Anderson, SC (US); Clayton Culbreath, Anderson, SC (US)

(21) Appl. No.: 17/604,217

(22) PCT Filed: Apr. 16, 2020
(86) PCT No.: PCT/US2020/028561

§ 371 Date: Oct. 15, 2021

Related U.S. Application Data
(60) Provisional application No. 62/834,846, filed on Apr. 16, 2019.

(51) Int. Cl. B29C 64/118; A61F 5/08; B33Y 10/00; B33Y 40/20; B29C 64/379; B29C 71/04

(52) U.S. Cl. CPC B29C 64/118; A61F 5/08; B33Y 10/00; B29L 2031/753; B29C 64/379; B29C 71/04; B29K 2995/0059; B33Y 40/20

(57) ABSTRACT

Disclosed herein are methods of making and using compositions comprising medical devices, particularly medical devices made from resorbable polymers.

### CROSS-REFERENCE TO RELATED APPLICATION

This application claims the benefit under 35 U.S.C. § 119(e) of U.S. Provisional Patent Application No. 62/834,846, entitled BIODEGRADABLE NASAL SPLINT, filed Apr. 16, 2019, which is herein incorporated in its entirety for all purposes.

### TECHNICAL FIELD

The present disclosure relates to compositions comprising a medical device, for example, a nasal septum splint, and methods for making and packaging such medical devices.

### BACKGROUND

There are many medical conditions and their treatments that require maintaining fluid flow and/or an open conduit within specific regions of human, animal or other subject's anatomy. Additionally, in treatments of subjects, it is sometimes desired to provide beneficial compositions on or within a degradable substrate, or to provide support to anatomical structures, or to space apart or separate two or more of a subject's anatomical structures or surfaces. A common method for maintaining fluid flow and conduit maintenance in a subject is to use one or more stents. Stents have been used in many different anatomical regions, including, but not limited to cardiac stents, vascular (arterial and venal) stents, organ duct stents, nasal stents, lachrymal stents, ear drum tubes, and ostial stents. Stents may be used to repair an anatomical defect, or for support of an existing anatomical structure, or may be a temporary implant that is later removed or is resorbed. Stents generally comprise a tube-like structure as a portion or the entire stent, and the dimensions, compositions, and properties of a stent may be selected for applicability to the use and anatomical site where the stent is intended to be used.

What is needed are degradable or resorbable medical devices that are placed in, on or between one or more anatomical structures, for example, for at least one of anatomical support, fluid flow, conduit maintenance, separation, and/or providing at least a bioactive composition. The present disclosure provides degradable medical devices, for example a nasal stent, and methods of making and using degradable medical devices.

### SUMMARY

The present disclosure comprises methods and compositions comprising degradable polymeric medical devices that are manufactured using degradable polymeric materials and are formed into a stable medical device by contact with a force-applying and/or shape-maintaining mold, a force-applying and/or shape-maintaining container, and/or one or more force-applying and/or shape-maintaining components.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings.

FIG. 1 is a drawing of an exemplary mold that was used to form a nasal splint from a pre-form planar structure.

FIG. 2 is a drawing of an exemplary a force-applying and/or shape-maintaining container for forming a medical device, for example a nasal splint, from a pre-form, and is opened to show a nasal splint disposed therein.

FIG. 3 is a drawing of a cross-section of the exemplary a force-applying and/or shape-maintaining container of FIG. 2.

FIG. 4 shows an exemplary nasal splint disclosed herein.

FIG. 5 shows an exemplary nasal splint disclosed herein.

FIG. 6 shows an exemplary nasal splint disclosed herein.

FIG. 7A shows an exemplary planar preform. FIG. 7B shows an exemplary force-applying and/or shape-maintaining component, for example, a mandrel. FIG. 7C shows the planar preform of 7A contacting the force-applying and/or shape-maintaining component of 7B to shape a medical device, for example, a stent. FIG. 7D shows an exemplary structurally stable medical device, for example, an abdominal aortic stent.

FIG. 8A shows an exemplary force-applying and/or shape-maintaining container for making a single layer corrugated medical device. FIG. 8B shows an exemplary force-applying and/or shape-maintaining container for +making a double layer corrugated medical device. FIG. 8C shows a preform (dark) in position in an exemplary force-applying and/or shape-maintaining container (shown as transparent) for making a double layer corrugated medical device. FIG. 8D shows an exemplary double layer corrugated medical device.

### DETAILED DESCRIPTION

Disclosed herein are medical devices comprising degradable polymeric materials, and methods for making and using such medical devices. It is believed that medical devices disclosed herein provide increased compliance by subjects in maintaining the device in place, primarily because disclosed medical devices provide increased patient comfort compared to previously known medical devices. For example, exemplary disclosed nasal stents are more comfortable for a subject, leading to increased subject tolerance and potential for the nasal stent to be in place for a longer time, resulting in an enhanced outcome for the subject.

Disclosed herein are medical devices made with degradable polymeric materials that are manufactured by using additive manufacturing methods known in the art to produce a printed article, and forming the printed article into a structurally stable degradable medical device by contacting the degradable printed article (referred to herein as a pre-form) with a mold, a force-applying and/or shape-maintaining container, and/or one or more force-applying and/or shape-maintaining components. Degradable medical devices disclosed herein include, but are not limited to, one or more of a coronary vascular stent; a vascular stent; a peripheral vascular stent; a carotid stent; a cerebral stent; a cell transportation device; a cell growth platform; a device for supporting an anatomical lumen; a device for reinforcing an anatomical lumen; a device for separating one or more anatomical structures or surfaces; a device for delivering a drug or drugs to an anatomical lumen or site; a renal stent; a iliac stent; a superficial femoral artery stent; a urethral stent; a ureter stent; a urinary stent; a biliary stent; an implantable scaffold; a tracheal stent; a trachea stent; a large bronchi stent; a nasal stent; a gastrointestinal stent; an esophageal stent; a drug delivery stent; a drug delivery device; a self-expandable stent; a balloon-expandable stent; a coil stent; a helical spiral stent; a woven stent; an individual ring stent; a ratcheting stent; a modular stent; a bifurcated stent; a stent-graft; a graft; a birth control device; an intrauterine device (IUD); an anatomical lumen repair or splicing device; a device for local delivery of active ingredients to anatomical sites, for example a tubular shaped lumen or organs, for treatment of cancer, or medical or surgical repair, or corrective procedures; a device for treatment of colon or rectal cancer; an implant; a patch; a mechanical support device; a reinforcement device; a repair device; an attachment device; an oncology treatment device; a device for treatment of cancer within or near an anatomical site, such as a lumen or anatomical structure's surface or interior; a device to assist in remodeling of diseased anatomical lumens; a tissue engineering application (for example, for bone, cartilage, blood vessels, bladder, skin, muscle, etc.); a bone fixation device; bone plates; a medical textile; a repair, a device for reconstruction, or replacement/repair of ligaments; a device for maxillofacial surgery; a device for repair, reconstruction, or replacement of rotator cuffs; a device for repair, reconstruction, replacement of hollow organ tissue; a screw; a plate; any implantable devices or patches for regenerative medicine; and a device for the treatment of cancer or other pathologies of a subject.

As an exemplary device, disclosed herein are methods for making a degradable nasal splint or nasal stent. It is intended that this disclosure is not limiting to the understanding of methods of making degradable medical devices, such as those included above. Additive manufacturing methods are known and can be used to “print”, using degradable polymeric materials, a planar pre-form disclosed herein. A planar pre-form can be formed into a stable structure that may be the intended final form of the degradable medical device or the stable structure may undergo further treatments such as coatings, sterilization, or treatments that alter the degradation rate of the degradable medical device. As used herein, a nasal stent and a nasal splint may be used interchangeably to refer to a disclosed medical device that provides at least one of support, conduit maintenance and/or fluid flow for the paranasal sinuses, including the maxillary sinuses, frontal sinuses, ethmoid sinuses and sphenoid sinuses, the nasal or sinus ostia, the sinus cavities and nasal structures such as external meatus, external nostrils, septum and nasal turbinates. Such medical devices may be used in surgical treatments for nasal-related treatments and/or repair.

In an aspect, degradable medical devices disclosed herein, such as nasal splint devices and pre-form nasal splint devices disclosed herein, comprise a composition comprising one or more degradable polymeric materials. Degradable, also known as resorbable, biodegradable, bioresorbable, and like terms may be used interchangeably, and mean “at least a portion of a material that is degraded or broken down into one or more constituents, for example, when exposed in a moist or biological environment, and includes any variety of mechanisms of degradation.” The rate of degradation that is found when the material is placed in an environment where it can degrade, for example, a biological environment, such as exposure to bodily fluids and/or temperatures, may be accelerated by manufacturing steps, such as exposure to ionizing radiation or chemical solutions, or by additives or materials incorporated into compositions comprising degradable polymers or coatings on at least a portion of fiber stream, a degradable pre-form and/or a disclosed degradable medical device. As used herein, polymers and copolymers refer interchangeably to polymeric materials comprising monomers wherein the monomer may have the same chemical formula (homopolymers) or differing chemical formulae (copolymers of two or more types of monomers). All or a portion of the material may bioresorb or degrade. For example, at least 50%, or, at least 60%, or at least 70%, or at least 80%, or at least 90%, or at least 95%, or at least 97.5%, or at least 98%, or at least 99%, or at least 99.5%, or at least 99.9% of the mass of a material used to form a degradable medical device may degrade within a suitable period of time placement in an anatomical site.

Time periods for degradation of a substantial portion of a medical device disclosed herein may be dependent on the type and amount of the one or more degradable compositions, such as polymeric materials, used to make a degradable medical device. Additionally, the structure and/or porosity of a disclosed medical device may affect the time until a degradable device loses at least a portion of its structural integrity. In general, it is contemplated that a medical device disclosed herein may degrade and lose structural integrity, for example lose a portion of its ability to provide structural support to an anatomical site in a time period of from about 1 week to about 24 weeks, from about 2 weeks to 4 weeks, from about 1 week to 4 weeks, from about 1 week to about 6 weeks, from about 1 week to about 8 weeks, from about 2 weeks to about 8 weeks, from about 1 week to about 12 weeks, from about 2 weeks to about 10 weeks, from about 3 weeks to about 10 weeks, from about 1 week to about 16 weeks, from about 2 weeks to about 12 weeks, from about 3 weeks to about 10 weeks, from about 1 week to about 20 weeks, from about 2 weeks to about 18 weeks, from about 3 weeks to about 15 weeks, from about 1 week to about 24 weeks, from about 2 weeks to about 22 weeks, for less than 1 week, for longer than 24 weeks, and for ranges and days thereinbetween.

Degradable polymers for use in additive manufacturing are suitable for the present invention. Polymeric materials disclosed herein comprise polymers or copolymers comprising monomer or oligomeric subunits (e.g. residues), comprising, but not limited to, monomeric or polymeric residues comprising L,L-lactide, D,L-lactide, glycolide, substituted glycolides, para-dioxanone, 1,5-dioxepan-2-one, trimethylene carbonate, epsilon-caprolactone, alpha-Angelica lactone, gamma-valerolactone and delta-valerolactone; glycolic acid; ethylene glycol; hydroxy-alkanoate; caprolactone; orthoesters; phosphazene; polyesters, polyether esters, hydroxybutyrate; polycarbonate, trimethylene carbonate; esteramides; anhidrides; dioxanone; alkylene alkylate; biodegradable urethane; etheresters; acetals; succinimides; sebacic acid, adipic acid, terephthalic acid; imino carbonates; phosphates, polyphosphonates, polyphosphazenes; poly(lactide); poly(glycolide); poly(lactide-co-glycolide); poly(lactic acid); poly(glycolic acid); poly(lactic acid-co-glycolic acid); poly(lactide)/poly(ethylene glycol) copolymers; polyglycolic acid (PGA), polylactic acid (PLA), lactic acid-glycolic acid copolymer (PLGA), polyhydroxyalkanoates (PHA), polyhydroxybutyrate-valerate (PHBV), polyvinyl alcohol (PVA), polyethylene terephthalate (PET), polyglycolide-lactide, polycaprolactone (PCL), lactic acid-E-caprolactone copolymer (PLCL), polydioxanone (PDO), polytrimethylene carbonate (PTMC), poly(amino acid), polydioxanone, polyoxalate, a polyanhydride, a poly(phosphoester), polyorthoester and copolymers thereof, poly hyaluronic acid; poly(glycolide)/poly(ethylene glycol) copolymers; polyether-ester polymers, poly(para-dioxanone).a polyhydroxy-alkanoate, poly(lactide-co-glycolide)/poly(ethylene glycol) copolymer; poly(lactic acid)/poly(ethylene glycol) copolymer; poly(glycolic acid)/poly(ethylene glycol) copolymer; poly(lactic acid-co-glycolic acid)/poly(ethylene glycol) copolymer; poly(caprolactone); poly(caprolactone)/poly(ethylene glycol) copolymer; poly(orthoester); poly(phosphazene); poly(hydroxybutyrate) or copolymer including a poly(hydroxybutyrate); poly(lactide-co-caprolactone); polycarbonate, poly(trimethylene carbonate); polyesteramide; polyanhydride; poly(dioxanone); poly(alkylene alkylate); copolymer of polyethylene glycol and a polyorthoester; biodegradable polyurethane; poly(amino acid); polyetherester; polyacetal; polycyanoacrylate; poly(oxyethylene)/poly(oxypropylene) copolymer, polysuccinimide; a polyanhydride poly(sebacic acid), poly(adipic acid), poly(teraphthalic) acid; polyamide; poly(imino carbonate) polyamino acid; phosphorus-based polymer; polyphosphate, polyphosphonate, or polyphosphazene; or combinations thereof. For example, a polymeric material may comprise a copolymer comprising glycolide residues, trimethyl carbonate residues, and caprolactone residues.

Degradable polymers and copolymers used herein may have the same or different chemical composition, or polymers and copolymers may have the same chemical composition, e.g., the same monomers, but such monomers are polymerized in different arrangements, e.g., random polymerization or block polymerization, or in different concentrations of monomeric components. Copolymers and polymers may be made by ring-opening polymerization, a process that is known to those of skill in the art.

As is known in the art of additive manufacturing, a liquid polymeric composition is used to print a structure. A liquid polymeric composition may comprise degradable polymers that are extruded through a print head to form a fiber (also referred to herein as a fiber stream), for example, in fused filament deposition, or a liquid polymeric composition may comprise monomers that may be polymerized by exposure to radiation, such as UV light.

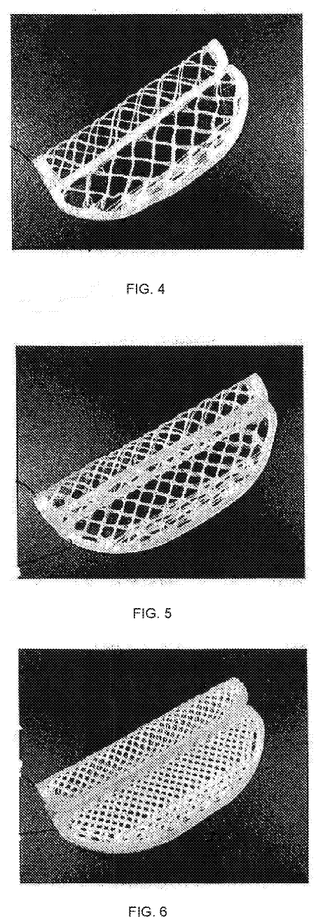

In an aspect, an exemplary medical device, for example, a nasal splint, such as those shown in FIGS. 4, 5 and 6, comprises a structure made of one or more degradable polymeric compositions printed as a continuous or discontinuous degradable copolymer fiber, made by additive manufacturing methods disclosed herein, comprising glycolide, trimethyl carbonate, and caprolactone monomeric residues. For example, a degradable copolymer may comprise from about 50 mole percent to about 60 mole percent glycolide, from about 20 mole percent to about 30 mole percent trimethyl carbonate, and from about 10 mole percent to about 30 mole percent caprolactone, of the total number of moles of the copolymer. For example, a degradable copolymer may comprise from about 50 mole percent to about 60 glycolide mole percent, from about 20 mole percent to about 30 trimethyl carbonate mole percent, and from about 10 mole percent to about 30 mole percent caprolactone, of the total number of moles present within the copolymer. As shown in FIGS. 4, 5 and 6, a nasal splint is made by additive manufacture that comprises using a 3-D printing apparatus utilizing a degradable polymeric material to form a lattice structure, followed by subsequent printing of one or more layers of degradable polymeric material fibers.

### Method of Making

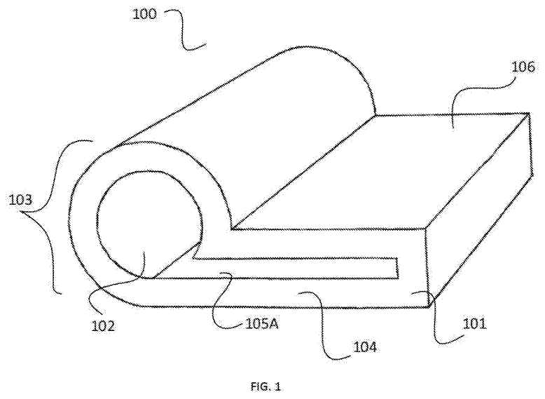

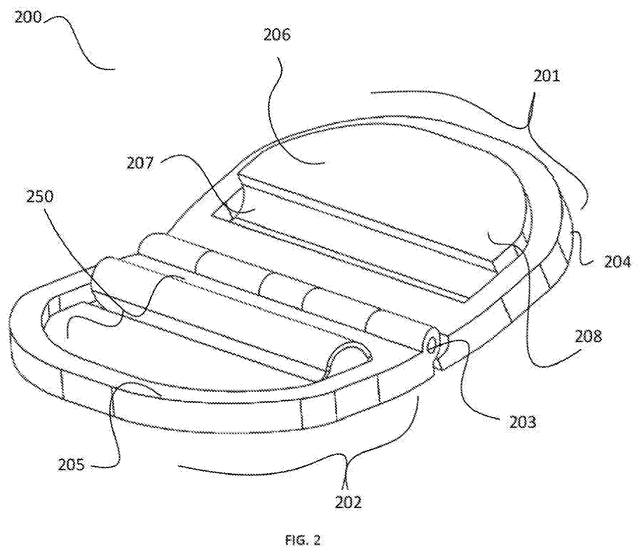

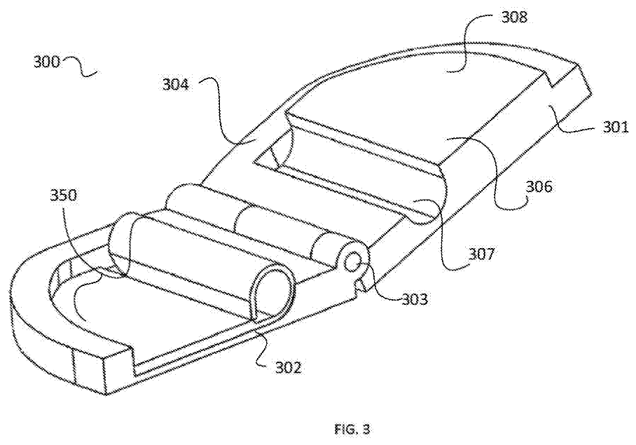

Disclosed herein are methods of making degradable medical devices using additive manufacturing methods. In general, a method comprises a) manufacturing or printing a pre-form with a 3-D printing (additive manufacture) device using one or more degradable 3-D printable polymeric fibers and b) forming the pre-form into a structurally stable medical device or a structurally stable portion of a medical device. A medical device disclosed herein may comprise a medical device or may be used as a degradable component in a medical device, which may or may not be degradable. A pre-form is formed into a structurally stable medical device by shaping the pre-form medical device using a force-applying and/or shape-maintaining container, and optionally one or more force-applying and/or shape-maintaining components. Such a force-applying and/or shape-maintaining container may be, for example, a stand-alone mold into which the pre-form is entered for a predetermined time or the force-applying and/or shape-maintaining container may be a packaging container that can shape or form one or more pre-forms into a structurally stable medical device and serve as a packaging container for shipping and storage of one or more structurally stable medical device. See FIG. 1 for an exemplary mold and FIGS. 2 and 3 for a container that when closed will form, for example, a nasal splint. Not shown in FIGS. 2 and 3 is a mandrel (a cylindrical rod around which material is shaped) that may be placed within the tube portion formed from the pre-form to aid in maintaining the lumen of the tube formed. One of skill in the art can design force-applying and/or shape-maintaining containers and force-applying and/or shape-maintaining components to shape a degradable pre-form into a desired shape.

In an aspect, a structurally stable nasal splint is disclosed herein. Structurally stable means that a planar pre-form has been shaped into a medical device and the structure is in its final conformation, though the structure may undergo post-treatments to result in the final medical device. It is expected that personnel using a disclosed medical device, in fitting the medical device for a particular subject, may make adjustments to the final conformation of the medical device, such as trimming a portion of the medical device to fit an anatomical region of the subject. The final conformation of a medical device is meant to refer to the medical device as it is ready for combination with another structure (when a disclosed medical device is a component of a medical device), or is shipped from manufacturing to be placed in commerce for, or given to, end users.

Disclosed herein is a degradable pre-form comprising a planar body having a top and bottom surface, and an edge formed by the material thereinbetween the top and bottom surfaces. In an aspect, a planar body may have a shape that is a rectangle, optionally having one or more rounded corners. In an aspect, a planar body may have a shape that can be any planar geometrical or non-geometrical shape, including, but not limited, to a circle, a star, a triangle, a square, a parallelogram, an octagon, or a rhomboid and/or random undefined shapes. A polymeric planar body may be of a shape that will be complementary to and fit in the intended site of placement in a subject. A planar body disclosed herein made also be referred to as a pre-form.

An exemplary method for making a medical device disclosed herein may comprise contacting a planar pre-form with a force-applying and/or shape-maintaining mold, a force-applying and/or shape-maintaining container and/or one or more force-applying and/or shape-maintaining components. In an aspect, a medical device may be formed by contacting a pre-form with one or more force-applying and/or shape-maintaining components to shape a planar pre-form into its final conformation. A method disclosed herein comprises shaping a pre-form nasal splint into a nasal splint by confining the planar body in a mold or container disclosed herein. Such a mold or container may be also be used as a packaging material for one or more nasal splints, and result in fewer steps needed in manufacturing, packaging, shipping, and/or storage.

Disclosed herein are degradable pre-form nasal splints comprising a planar body having a top and bottom surface, and an edge formed by the material thereinbetween the top and bottom surfaces. In an aspect, the planar body may have a shape is a rectangle, optionally having one or more rounded corners. In an aspect, the planar body may have a shape can be any planar geometrical or non-geometrical shape, including, but not limited, to a circle, a star, a triangle, a square, a parallelogram, an octagon, or a rhomboid and/or random undefined shapes. The shape of the polymeric planar body may be optimized to fit the surrounding anatomy of the intended site of placement in a subject. A planar body disclosed herein made also be referred to as a pre-form nasal splint.

Disclosed herein are pre-form and shaped medical devices comprising one or more degradable polymeric materials disclosed herein. A planar body may be made by additive manufacturing methods, including, but not limited to, fiber-deposition manufacture using a 3-D printing apparatus and forming one or more types of degradable polymeric fibers. Other types of 3-D printing methods, including, but not limited to SLA, laser sintering and CLIP, and appropriate polymeric materials may be used to form medical devices disclosed herein.

For example, a method of additive manufacturing is fused filament fabrication (FFF). The majority of additive manufacturing through FFF utilizes a single-phase thermoplastic polymeric monofilament to generate a print line through melt extrusion. The print line is in a horizontal plane, which may be referred to as a plane in the x-y direction, and that x-y plane may contain independent multiple print lines, depending on the desired design of the article. Sometime, multiple articles are printed at the same time, in which case multiple first print lines area laid down in a single (first) x-y plane. In order to create a 3-dimensional article, i.e., in order to create an article having a z-direction, one or more second print lines are laid down in a second x-y plane that sits on top of the first x-y plane defined by the location of the first print line(s). The height of the printing, i.e., the extent of z-direction, is defined by the number of x-y planes that are printed on top of one another. The printhead(s) and printing by a 3-D printer are directed by a software program, and such software programs and directions for printing using a 3-D printer are known to those of skill in the art.

In a method of making a nasal splint, the degradable polymeric planar body, (a pre-form), is further manipulated so as to form a nasal splint comprising a substantially planar structure with a tubular portion formed by at least a portion of the planar structure. For example, a tubular structure is made by moving at least a portion of the bottom surface and its edge of the planar body is moved upward and curled inward toward the top surface to form tube-like structure as the edge approaches the top surface or another portion of an edge of the planar body. The moved edge may or may not physically contact the top surface or another portion of an edge. For example, in a rectangle planar body of a pre-form nasal splint, one of the two short sides of the rectangle is rolled upwardly and curved inwardly so that at least a portion of the back surface is raised apart from and parallel to the front surface to form a tube-like structure on one end of the rectangle. The edge of the short side may contact the front surface to form a connected, unbroken tube (continuous tubular wall) structure or the edge of the short side may not contact the front surface to form a slit tube (discontinuous tubular wall) structure. In the above description, reference to continuous or discontinuous refers to the wall of the tube, and not to the lumen of the tube formed by the wall, which has unobstructed openings on both ends of the tube (formed by the longer sides of the rectangle) wherein the ends are open and the tube is hollow therethrough its lumen. This tubular structure forms a conduit for fluid flow, such as liquids or air, as well as providing anatomical support to maintain separation between and support of tissue structures. The tubular structure's walls may form a lumen that is uniform in width throughout its longitudinal axis or the lumen width may vary uniformly, such as in a cone shape, or may vary randomly in width along the longitudinal axis. Such a nasal splint is shown in FIGS. 4, 5 and 6. One or more sides of the rectangle may have rounded corners.

A planar body pre-form nasal splint may be formed into a nasal splint comprising a tubular portion by contacting the planar body with a mold, mold-like container, which may be a packaging container, for one or more nasal splints. See FIGS. 1 and 2, and a side cutaway view in FIG. 3. FIG. 1 shows a mold for a nasal splint 100. Mold 100 comprises one or more walls 101 that define an interior space 102. A pre-form nasal splint, with its planar-shape body, is formed so that it conforms to interior space 102. For example, in using mold 100, a planar rectangle pre-form nasal splint is shaped to conform to interior space 102 by rolling a short edge of the rectangular planar pre-form to form a hollow tube made from the rolled edge and inserting it into tubular section 103 of mold 100. Concurrently, the remaining planar section of the rectangular planar pre-form is inserted into planar section 104 of mold 100. The rolled pre-form may be inserted into either of the open sides 105 a/b (b is not shown in FIG. 1, but b is opposite a) or mold 100 may be hinged so as to open top section 106 for insertion of the pre-form into interior space 102 (hinge not shown in FIG. 1) Mold 100 is a force-applying and/or shape-maintaining container in that mold 100 prevents the rolled end of the planar shape body from unrolling and maintains the planar shape of the unrolled portion of the pre-form. Optionally one or more force-applying and/or shape-maintaining components may be used. For example, and not shown, a mandrel may be placed within the tubular opening formed by the rolled portion of the pre-form to maintain the open lumen of the roll. Alternatively, a small rod component may be placed between the outside of the rolled portion and the inside surface of mold 100 to tension hold the rolled portion in place. As noted above, the rolled portion of the pre-form may contact the top surface of the planar shaped pre-form body entirely across the entire edge, to form a continuous wall tube or may only contact a portion of the top surface, or may be proximate to the surface so that an open space exists between the edge and the top surface and forms a discontinuous wall tube.

FIG. 2 shows an exemplary force-applying and/or shape-maintaining container 200 with nasal splint 250 therein. Container 200 has top section 201 and bottom section 202. As shown in FIG. 2, top section 201 and bottom section 202 are moveably connected by hinge 203, so that top section 201 can hinge forwardly and contact bottom section 202 to form a closed container 200. Alternatively, and not shown, top section 201 and bottom section 202 could be separate components and not joined by hinge 203. In such alternatives, top section 201 and bottom section 202 could be positioned so that top edge 204 contacts bottom edge 205 to form a closed container 200. In such alternatives forming a closed container 200, top section 201 and bottom section 202 could maintain a closed container by contact due to gravity, or by snap fit contact elements, clips or other restraining elements that maintain contact between top section 201 and bottom section 202.

As shown in FIG. 2, interior surface 206 is shaped to form concave area 207 that is shaped to receive the rolled portion of a nasal splint 250. Optionally, a portion of interior surface 206 may be raised upwardly to extend above edge 204 to form insertion portion 208. When top portion 201 is moved to contact bottom portion 202 so as to form a closed container 200, insertion portion 208 provides force-applying and/or shape-maintaining functions for the pre-form nasal splint planar portion to remain planar while within container 200. Insertion portion 208 may or may not contact a planar portion of the pre-form nasal splint planar portion, and when contacting provides a force-applying function, and when not contacting, provides a shape-maintaining function.

In FIG. 3, a cross-section view of the exemplary container of FIG. 2 is shown, and like numbers indicate like structures. Container 300 has top section 301 and bottom section 302. As shown in FIG. 3, top section 301 and bottom section 302 are moveably connected by hinge 303, so that top section 301 can hinge forwardly and contact bottom section 302 to form a closed container 300. As shown in FIG. 3, interior surface 306 is shaped to form concave area 307 that is shaped to receive the rolled portion of a nasal splint 350. Optionally, a portion of interior surface 306 may be raised upwardly to extend above edge 304 to form insertion portion 308. When top portion 301 is moved to contact bottom portion 302 so as to form a closed container 300, insertion portion 308 provides force-applying and/or shape-maintaining functions for the pre-form nasal splint planar portion to remain planar while within container 300.

FIGS. 4-6 each show a nasal splint made by methods and containers disclosed herein. In general, methods for making a nasal splint comprise a) making a pre-form planar body and b) forming the pre-form planar body into a nasal splint by contacting the pre-form planar body with a mold or container to form the final nasal splint form. Such as formed nasal splint may be sterilized or may be provided in a non-sterile status.

Methods for making a degradable medical device or component may comprise the following steps.

Making a planar shape pre-form comprises loading one or more degradable polymeric filaments (polymeric materials), for example, degradable polymeric fibers, in a 3-D printing device. As described herein, the 3-D printing device is described as filament deposition printing, but one of skill in the art can understand that any known additive manufacturing methods can be used to make pre-forms. Optionally, the filament may be conveyed into the 3-D printers' printing head through a sealed Teflon tube to minimize exposure to contaminants and moisture. The printing head is held at a temperature that will facilitate printing with the filaments used, such as from 150 to 350° C. The printing head may be a plurality of printing heads allowing for printing of a plurality of pre-forms. The printing head may be a dual direct drive print head for flexible materials. The printed fibers (or fiber streams) are deposited in a predetermined pattern, according to the software directing the printer, onto a printing bed or build plate. The printing bed or build plate can be at room temperature and may be a metal plate, optionally with a smooth surface, such as a mirror surface finish, and may have a coating applied to the build plate. Other build plate materials, finishes, and coatings on the build plate are intended in the disclosed methods to achieve optimal surface energy and heat capacity/conductivity for differing degradable polymeric materials, and the examples provided herein are not intended to be limiting.

For example, in making a nasal splint disclosed herein, printed by a 3-D printing apparatus, wherein a print head, following predetermined instructions from the controlling software printed, on the build plate, a rectangular shape, having one short side with rounded corners and one short side with perpendicular corners (straight edge), with a molten or liquid fiber stream from the print head. Once the entire rectangle was formed, the print head moved to print a second rectangle inside the first rectangle so as to form a two-fiber width rectangular edge. The print head then printed, at approximately a 45 degree angle, rectilinear fiber streams in the interior space of the rectangular edge. The edge fibers and interior rectilinear fiber streams were each in the same plane so that one layer was formed. The % infill for the interior of the first layer was about 20%, and the same % infill for further layers of rectilinear fiber streams.

After printing the first layer, the printing head was positioned to lay a second layer of fiber streams on top of the first two edge fibers forming the outer perimeter of the rectangle. After forming the 2^{nd} layer's two edge fiber streams, the printer head moved to print 45 degree rectilinear fiber streams in the interior of the rectangular edges of the second layer, orthogonally to the first layer, to form a second layer of rectilinear fibers so that the two printed layers form a cross-hatched pattern. FIGS. 4-6 show differing number of rectilinear fiber streams in a cross-hatching pattern used for each nasal splint illustrated in FIGS. 4-6. The edge fibers and interior rectilinear fiber streams are each in the same plane so that one layer is formed. The spacing between each fiber stream is determined by the % infill desired for the interior of the rectangle shape. In disclosed nasal splints herein, % infill can range from about 0% to about 100%, from about 5% to about 90%, from about 10% to about 80%, from about 20% to 70%, from about 30% to 60%, from about 40% to about 50%, from about 10% to about 50%, from about 5% to about 20%, from about 10% to about 30%, from about 5% to about 15%, from about 2% to about 20%, from about 10% to about 15%, from about 20% to about 30%, from about 80% to about 100%, and all ranges therein between.

After forming the first layer, the printing head is positioned to print a second layer of fiber streams on top of the first two edge fibers forming the outer perimeter of the rectangle. In an aspect, after forming the 2^{nd} layer's two edge fiber streams, the printer head moves to print rectilinear fiber streams at a 45 degree angle from the inner edge fiber in the interior of the rectangular edges, orthogonally to the first layer's fiber direction, to form a second layer of rectilinear fibers. Together, these two layers create a cross-hatching pattern of fibers. This pattern can be repeated to add one or more further layers. For example, a nasal splint may comprise from 2 to 12 layers, from about 2 to 20 layers, from about 5-10 layers, and ranges therein between. Those of skill in the art can determine how many layers are needed to form a suitable nasal splint or other types of medical devices.

For example, one or more portions of a disclosed planar preform can have differing numbers of layers. For example, one portion of a planar preform may have fewer layers than one or more portions of the preform. For example, one portion of a preform may have more layers than one or more other portions of the preform. For example, in the structurally stable medical device, for example a nasal splint, the portion of the preform that is shaped into a tubular portion may have more layers that than does the planar section of the nasal splint. For example, in the structurally stable medical device, for example a nasal splint, the portion of the preform that is formed into a tubular portion may have fewer layers that than does the planar section of the nasal splint.

One or more layers of a planar preform may have the same or different pattern of printing the fiber stream. In the above nasal splint example, at least the first and second layers each have cross-hatched, or rectilinear, fiber streams that are orthogonally displace from the other layer. Subsequent layers could have the same or different patterns of fiber deposition. Alternatively, a first layer could have one pattern and a second or subsequent layer could have the same pattern or a different pattern.

Pores are formed in a planar preform between the fibers within a layer and between layers of printed fibers, such as a cross-hatch pattern or other patterns formed. Such pores may aid in tissue in-growth or release of bioactive materials from the medical device.

As used herein, fiber stream may refer to using a continuous stream of polymeric material through the print head to form an entire planar structure with no interruptions in the fiber stream so that the planar structure comprises one continuous fiber, or a fiber stream may refer to one or more discontinuous fibers, formed by a stop in a fiber stream and then restarting the fiber stream at the same position or a different position. A fiber stream may comprise one polymeric material or may switch from one polymeric material to a second or more polymeric material, and can return to printing with the first or subsequent polymeric materials. It is understood that a fiber stream as used herein may be referred to a fiber or a deposited fiber.

Those skilled in the art understand that printing instructions are input into the controller elements of the 3-D printing device. A disclosed medical device may be made with one or multiple layers of deposited fibers. Each lay may be identical to the layer above or below it, or may be different as to polymeric composition, coatings, thickness (height), or other characteristics. For example, a rectangular planar pre-form nasal splint can be formed by the following: first layer height 0.3 mm, and 0.2 mm second and other layers that range from 0.05 mm to 5 mm in thickness. Print speed may be the same or different for each layer printed. Those of skill in the art can determine a print speed that yields acceptable products at a particular speed of printing.

Once the planar body (preform) is printed, it is removed from the build plate. For example, the planar body can be peeled from the build plate. For example, the build plate and the planar body that was printed, may be cooled, for example to from 5 to 10° C., so that the planar body can be removed from the build plate surface.

Though not wishing to be bound by any particular theory, it is believed that forming the planar pre-form into a structurally stable disclosed medical device, relies on the crystallization network formed by the degradable polymeric material. For example, in generally known methods for molding plastic materials, the molded plastic shape is maintained by internal stresses comprised in primarily the amorphous sections of the plastic material. In contrast, degradable polymeric materials disclosed herein may have amorphous and crystalline sections, and it is the crystallization networks, formed as the polymeric material crystallizes, that maintain the shape. Polymeric materials useful herein may have differing percentages of crystallization, which may affect the time needed to form a structurally stable medical device.

For example, Example 1 shows percentage of crystallization for forming a nasal splint from a planar preform. A pre-form printed from a degradable polymeric material may be shaped into a structurally stable medical device in a time period in which 20% to 100% of the crystallization of the degradable polymeric material occurs. A structurally stable medical device formed from planar preform may maintain its structurally stable shape after a percent crystallization of the degradable polymeric material from about 10% to about 100%, from about 20%, from about 30%, from about 40% from about 50%, from about 60%, from about 70%, from about 80%, from about 90%, from about 90% to 100%, and all ranges thereinbetween. Those of skill in the art can determine a percent crystallization of disclosed degradable polymeric materials. Such crystallization determinations and degradable polymeric materials suitable for use herein include, but are not limited to, those disclosed in PCT/US2020/021499 which is herein incorporated in its entirety.

For example, degradable polymers for use in additive manufacturing are suitable for the present invention, and include, but are not limited to, degradable copolymers and polymers disclosed in PCT/US2020/021499. In an aspect, a monofilament fiber used for methods herein comprises a polyaxial polymer of a formula M(B)2 or M(B)3, where M comprises repeating units and B comprises repeating units. Repeating units are monomers comprising, but not limited to, those disclosed herein. For example, in a polyaxial polymer, a majority of the repeating units in M may be polymerization residues from TMC (trimethylene carbonate and/or CAP (e-caprolactone) and a minority of the repeating units in M are the polymerization residues from LAC (l,l- or d,l-lactide and)/or GLY (glycolide), while in contrast, a majority of the repeating units in B are the polymerization residues from GLY and/or LAC and a minority of the repeating units in B are the polymerization residues from TMC and/or CAP. In this way, the mid-block M has properties resulting primarily from the presence of residues of TMC and/or CAP, influenced by a minor amount of the residues from LAC and/or GLY, while the end grafts B have properties resulting primarily from the presence of residues of LAC and/or GLY, influenced by a minor amount of the residues from TMC and/or CAP. Optionally, M comprises repeating units from both of TMC and CAP, so that M is a copolymer comprising a majority of a mixture of CAP and TMC residues as repeating units, as well as GLY and/or LAC derived repeating units as a minor proportion of the repeating units.

For example, the present disclosure comprises polymeric material comprising a monofilament fiber comprising a polyaxial polymer of a formula M(B)2 or M(B)3, where M may be a homopolymer or a copolymer, and comprises a plurality of repeating units, where at least 50 mol %, e.g., 70 mol %, of the repeating units in M are a polymerization product of at least one of trimethylene carbonate and epsilon-caprolactone; and B may be a homopolymer or a copolymer, and comprises a plurality of repeating units, where at least 50 mol %, e.g., 70 mol %, of the repeating units in B are a polymerization product of at least one of glycolide and lactide, and optionally both of glycolide and lactide In one embodiment, M is a copolymer.

Forming a structurally stable nasal splint from the planar pre-form may comprise the following steps. Depending on the environmental temperature at which the pre-form is kept, and on the crystallization percentage of the polymeric material used, there is a time period for forming a medical device, such as a nasal splint after printing the pre-form. For example, it is desirable that the pre-form is shaped to form the planar preform into other shapes, such as forming a tubular portion in a planar pre-form for a nasal splint, within 2 to 15 hours after printing, and the time may be shorter or longer depending on the degradable material used to make the pre-form and the environmental temperature the pre-form is stored in prior to placing it in the mold. It is desirable that a pre-form is shaped, such as rolled to form a tubular portion, within 2 to 15 hours after printing, within 4-12 hours after printing, within 6-10 hours after printing, and all times thereinbetween.

A rolled portion of a planar pre-form is formed by placing a planar pre-form in a force-applying and/or shape-maintaining mold or container disclosed herein. A force-applying and/or shape-maintaining container may be designed to form one or multiple pre-form planar bodies into one or multiple medical devices. A pre-form may also be contacted by one or more force-applying and/or shape-maintaining components that aid in maintaining the position and/or structure of a shaped portion, the planar portion or other portions of a pre-form body independent of or while in a mold or container.

After placement of a pre-form in a force-applying and/or shape-maintaining mold or container, the pre-form body may be visually inspected for completeness of the one or more shaped sections, the regularity of the shaped section(s), and for a nasal splint disclosed herein, separation or no separation between the rolled edge and the planar top section of the nasal splint. In post-printing storage, the contained (in a structurally stable medical device mold or container) pre-forms are stored to allow for setting the shape and if present, for onset of crystallization of the polymeric material. Such post-printing storage conditions may include storage at room temperature and moisture-free atmosphere for at least one day, at least two days, at least three days, at least four days, at least 5 days, from 1 to 2 days, from 1 to 3 days, from 0.5 to 5 days, from 0.5 to 10 days, and all times inclusive and therein between the ranges.

After completion of post-printing storage, a medical device may be removed from a force-applying and/or shape-maintaining mold, placed into a container and further packaged. Alternatively, the force-applying and/or shape-maintaining container comprising at least one nasal splint contained within a disclosed container may be packaged. For example, packaging may comprise placing a force-applying and/or shape-maintaining container comprising one or more nasal splints under vacuum conditions for a predetermined time period, such as 0.5 to 3 days, before placing the force-applying and/or shape-maintaining container into a packaging container such as a foil pouch, and sealing the pouch. One or more multiple pouches may be placed in shelf boxes for storage, shipping, or further post-treatment such as sterilization.

### Post-Treatment of Medical Devices Disclosed Herein

A post-treatment of a medical device, such as a nasal splint, may comprise sterilization of one or more medical devices. Medical devices, such as nasal splints, may be provided in a sterile or non-sterile condition. Non-sterile medical devices, such as nasal splints, may be used in methods known by those of skill in the medical arts. Sterile medical devices, such as nasal splints, may be used in methods known by those of skill in the medical arts. Sterilization of one or more medical devices, such as nasal splints, may be accomplished by known methods for sterilizing medical devices such as gamma radiation, e-beam (electron beam), ethylene oxide, x-ray, heat, chemicals, nitrogen dioxide, irradiation, high pressure and filtration like steam under pressure, dry heat, ultraviolet radiation, gas vapor sterilants, chlorine dioxide gas, supercritical CO₂, and other known methods of sterilization.

Exposing a contained medical device, such as a nasal splint, to ionizing radiation, which can sterilize a medical device, such as a nasal splint, can also aid in enhancing degradation of the polymeric materials of the medical device, such as a nasal splint. For example, electron beam conditions can enhance degradation of polymeric materials. In an aspect, degradable polymers of a medical device, such as a nasal splint, may be subjected to post-processing, for example, in order to modify one or more characteristics of a degradable polymer. For example, in some embodiments, a degradable polymer may be exposed to a radiation dosage in order to modify the degradation profile of a degradable medical device, such as a nasal splint. In various embodiments, a degradable polymer may be exposed to ionizing energy such as E-beam irradiation or gamma radiation, for example, in a dosage of from about 1 kGy to about 100 kGy, or from about 30 kGy to about 60 kGy, or from about 35 kGy to about 45 kGy, from about 20 kGy to about 70 kGy, from about 10 kGy to about 80 kGy, from about 40 kGy to about 80 kGy, from about 50 kGy to about 70 kGy, from about 60 kGy to about 90 kGy, and all ranges and amounts thereinbetween. Not intending to be bound by theory, exposure of a degradable polymer of a medical device, such as a nasal splint, may be effective to impart a desired degradation profile to the medical device, such as a nasal splint, for example, such that the medical device, such as a nasal splint, exhibits loss of mechanical integrity within not more than about 4 weeks, more preferably within not more than about 2 weeks.

In an aspect, all or at least a portion of a pre-form body or structurally stable medical device, for example a nasal splint, may be coated with one or more polymeric coatings. A coating may be applied to a fiber prior to formation of the planar structure, to a planar structure (pre-form body), or to the structurally stable medical device, for example a nasal splint, formed after shaping the pre-form body. A coating may comprise, but is not limited to, one or more active agents, one or more cellular adhesion inhibitors or attractants, one or more hemostatic agents, may be the same polymeric material as a fiber stream of the planar structure, may be a different polymeric material from a fiber stream of the planar structure, may be partially or completely degradable, may be partially or completely nondegradable, may be liquid repelling, may be lubricious, or may be a combination of these or all of these. Such bioactive agents that can be used with disclosed medical devices are known to those of skill in the art.

### Methods of Using

For example, a structurally stable medical device, for example a nasal splint, made by methods disclosed herein may be used in surgical and non-surgical procedures known to those skilled in the art. For example, a method of use of a nasal splint may comprise positioning a disclosed degradable nasal splint comprising a tubular component at least partially defining a hollow passageway within subject's nasal passage between the nasal septum and an inferior turbinate. In a method of using a degradable medical device disclosed herein, a user may further alter the shape of the provided medical device by cutting or removing a portion of the degradable medical device. For example, a portion of the medical device may be removed so as to appropriately size the medical device for the anatomical site where the medical device is placed. It is to be understood that one or more medical devices can be used concurrently or sequentially to provide care to a subject.

### Kits

The present disclosure comprises a kit comprising a pre-form medical device and/or a structurally stable degradable medical device or component, disclosed herein, optionally contained within a force-applying and/or shape-maintaining mold, force-applying and/or shape-maintaining container and/or force-applying and/or shape-maintaining components disclosed herein, all of which may be provided within another packaging container. The kit may further comprise written instructions for its use.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, EIZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, when a compound is referred to as a monomer or a compound, it is understood that this is not interpreted as one molecule or one compound. For example, two monomers generally refers to two different monomers, and not two molecules.

As used herein, the terms “optional” or “optionally” means that the subsequently described event or circumstance can or cannot occur, and that the description includes instances where said event or circumstance occurs and instances where it does not.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, a mammalian subject is a human. The term “patient” includes human and veterinary subjects.

As used herein, the terms “administering” and “administration” refer to any method of providing a disclosed composition to a subject.

As used herein, the terms “comprises,” “comprising,” “includes,” “including,” “containing,” “characterized by,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. The term “comprising” may also include the limitations associated with the use of “consisting of” or “consisting essentially of”.

The transitional phrase “consisting of’ excludes any element, step, or ingredient not specified in the claim, closing the claim to the inclusion of materials other than those recited except for impurities ordinarily associated therewith. When the phrase “consists of’ appears in a clause of the body of a claim, rather than immediately following the preamble, it limits only the element set forth in that clause; other elements are not excluded from the claim as a whole.

The transitional phrase “consisting essentially of’ limits the scope of a claim to the specified materials or steps and those that do not materially affect the basic and novel characteristic(s) of the claimed invention. A ‘consisting essentially of claim occupies a middle ground between closed claims that are written in a ‘consisting of format and fully open claims that are drafted in a ‘comprising’ format. Optional additives as defined herein, at a level that is appropriate for such additives, and minor impurities are not excluded from a composition by the term “consisting essentially of’.

When a composition, a process, a structure, or a portion of a composition, a process, or a structure, is described herein using an open-ended term such as “comprising,” unless otherwise stated the description also includes an embodiment that “consists essentially of’ or “consists of’ the elements of the composition, the process, the structure, or the portion of the composition, the process, or the structure.

The articles “a” and “an” may be employed in connection with various elements and components of compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

The term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such.

The term “or”, as used herein, is inclusive; that is, the phrase “A or B” means “A, B, or both A and B”. More specifically, a condition “A or B” is satisfied by any one of the following: A is true (or present) and B is false (or not present); A is false (or not present) and B is true (or present); or both A and B are true (or present). Exclusive “or” is designated herein by terms such as “either A or B” and “one of A or B”, for example.

In addition, the ranges set forth herein include their endpoints unless expressly stated otherwise. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. The scope of the invention is not limited to the specific values recited when defining a range.

When materials, methods, or machinery are described herein with the term “known to those of skill in the art”, “conventional” or a synonymous word or phrase, the term signifies that materials, methods, and machinery that are conventional at the time of filing the present application are encompassed by this description. Also encompassed are materials, methods, and machinery that are not presently conventional, but that will have become recognized in the art as suitable for a similar purpose.

Unless stated otherwise, all percentages, parts, ratios, and like amounts, are defined by weight.

All patents, patent applications and references included herein are specifically incorporated by reference in their entireties.

It should be understood, of course, that the foregoing relates only to preferred embodiments of the present disclosure and that numerous modifications or alterations may be made therein without departing from the spirit and the scope of the disclosure as set forth in this disclosure.

The present disclosure is further illustrated by the examples contained herein, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present disclosure and/or the scope of the appended claims.

### EXAMPLES

### Example 1

A nasal splint was formed by printing a planar pre-form nasal splint and shaping the pre-form into a nasal splint comprising a tubular section and a planar section, as shown in FIGS. 4-6. The degradable polymeric material printed into the planar pre-form nasal splint comprised a degradable copolymer comprising from about 50 to about 60 glycolide mole %, from about 20% to about 30 trimethyl carbonate mole %, and from about 10% to about 30% caprolactone mole %, of the total number of moles present within the copolymer. The planar preform was made with a continuous fiber stream.

The planar preform was printed by a 3-D printing apparatus wherein a print head, following predetermined instructions from the controlling software printed, on the build plate, a rectangular shape, having one short side with rounded corners and one short side with perpendicular corners, with a molten or liquid fiber stream from the print head. Once the entire rectangle was formed, the print head moved to print a second rectangle inside the first rectangle so as to form a two-fiber width rectangular edge. The print head then printed, at approximately a 45 degree angle, rectilinear fiber streams in the interior space of the rectangular edge. The edge fibers and interior rectilinear fiber streams were each in the same plane so that one layer was formed. The % infill for the interior of the first layer was about 20%, and the same % infill for further layers of rectilinear fiber streams.

After printing the first layer, the printing head was positioned to lay a second layer of fiber streams on top of the first two edge fibers forming the outer perimeter of the rectangle. After forming the 2^{nd} layer's two edge fiber streams, the printer head moved to print 45 degree fiber streams in the interior of the rectangular edges of the second layer, orthogonally to the first layer, to form a second layer of rectilinear fibers so that the two printed layers form a cross-hatched pattern. A total of 15 layers were printed that repeated the patterns of layer 1 and 2. Those of skill in the art can determine how many layers are needed to form a suitable medical device.

The planar pre-form was removed from the build plate of the 3-D printing apparatus, and a tubular portion was shaped by wrapping the short side having perpendicular corners (straight edge) of the rectangular preform around a mandrel to form a tubular portion that curls inwardly toward the center of the top surface of the planar portion. The remainder of the planar preform stays planar. The tubular portion is shaped so that the straight edge is proximate to, but not contacting, the planar portion of the preforms top surface, i.e., the tubular portion has a space between its straight edge and the planar portion of the preform along its longitudinal axis. This shaped preform was placed in a force-applying and/or shape-maintaining container as shown in FIG. 2 in which the tubular portion and the planar portion of the preform were maintained, within a time frame of about 8 hours post-printing. After about 15 hours of contacting the force-applying and/or shape-maintaining container and component, the tubular portion and planar portion was stabilized (due to polymeric crystallization) to form a structurally stable nasal splint. From a determined crystallization curve shown in Table 1, it was calculated that at 8 hours approximately 50% crystallization had occurred, and at about 15 hours, 65% crystallization of the degradable polymeric material had occurred.

After formation of the structurally stable nasal splint, the nasal splint, still contained within the force-applying and/or shape-maintaining container was exposed to ionizing radiation of from about 30 kGy to about 60 kGy to enhance degradation of the polymeric material. Additionally, the nasal splint was sterilized by the irradiation. The force-applying and/or shape-maintaining container, with one or more structurally stable nasal splints therein, was packaged into foil pouches for shipping or storage.

### Example 2

### Stability of Formed Nasal Splint from Example 1

To determine the stability of structurally stable nasal splints prepared in Example 1, structurally stable nasal splints were placed in an incubator preheated to 50° C. The evaluation temperature was selected based on (1) shipping temperature excursion limits, (2) accelerated packaging considerations where 2 weeks at 50° C. simulates about 3 months of shelf stability at 22° C., and (3) a temperature above the glass transition temperature of the polymer. Splints placed in the incubator were removed from packaging and rested on a flat surface without additional support. At predetermined time points, splints were evaluated for visual changes in part shape. When evaluated at 13 days, no visual change in part shape or size, was observed. The tubular portion retained the set shape created during part formation.

### Example 3

### Subject-Matched 3D Printed Medical Device to Support Repair of an Abdominal Aortic Aneurysms

To create a medical device to aid in the repair of an abdominal aortic aneurysm, a custom stent can be prepared through additive manufacturing. First, the subject's aneurysm is imaged to create a point cloud image, which is then converted into a smoothed shell image. From the shell image, an analysis of the image results in the desired customized anatomical shape, which is then finalized as a solid body shell. The solid body shell is processed into a printing program via Fusion 360, and printed via Fused Filament Fabrication on a Hyrel Hydra printer using a PLA (polylactide polymer) filament. This printed element is the force-applying and/or shape-maintaining mold which is used to shape a planar preform into a structurally stabilized medical device.

The solid body shell image of the corrected and preferred physiologic shape is also used to generate a stent printing pattern by selecting, on the image points that can serve as the proximal and distal edges of the desired medical device. In the image, the solid body shell shape is “unrolled” to create a flat template, which is imported into Fusion 360 to generate a printing profile. A planar preform as described in Example 1 is printed. For example, two outline fiber streams and a rectilinear infill pattern at 20% infill is used as the pattern for each of the layers of the medical device. Layer height of 0.2 mm is designated, with a total thickness of 0.8 mm (4 layers). A Hyrel Hydra FFF printer is used with 0.4 mm diameter nozzle and 1.75 mm Lactoprene® 7415 filament (Poly-Med, Inc.) is the polymeric material. The planar pre-form was removed from the print bed before crystallization and immediately wrapped around the outside of the force-applying and/or shape-maintaining mold described above and held in place until at least 50% crystallization is complete.

Following crystallization, the structurally stable medical device, an endoprosthesis, can be removed from the force-applying and/or shape-maintaining mold, packaged in a tray, and sterilized for use in an implantation procedure to correct the abdominal aneurysm.

### Example 4

### Formation of a Degradable Medical Device to Provide Spacing Between Anatomical Surfaces or Structures

A degradable corrugated medical device is made by first printing a degradable polymeric planar preform, for example as taught in Example 1. Though not wishing to be bound by any particular theory, it is believed that 3-D printing a degradable polymeric planar structure, and then shaping that degradable polymeric planar structure in a force-applying and/or shape-maintaining mold or container, with or without one or more force-applying and/or shape-maintaining components, makes for a more stable 3-D printed medical device that maintains its structure or support until the polymeric material proceeds to degrade. For example, directly 3-D printing a curved tube (or other curved structure), wherein the 3-D does not result in a structure having the same stability as does curving a planar body into a curved tube. The risk for breakage of the directly printed curve is greater.

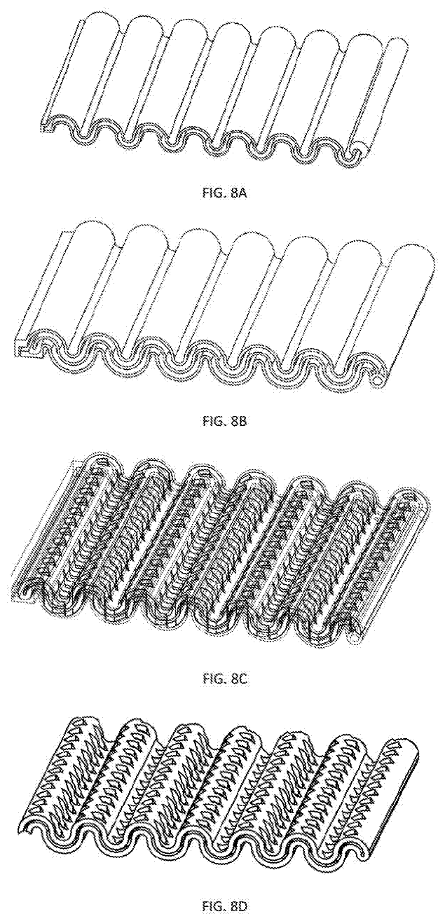

Once a planar preform is completed, it is removed from the 3-D printer's build plate and within the time period before at least 50% crystallization of the planar preform has occurred, the planar preform is placed into a force-applying and/or shape-maintaining container as shown in FIG. 8A or FIG. 8B. FIG. 8A shows a force-applying and/or shape-maintaining container in which a single layer corrugated degradable polymeric medical device is formed. The container of FIG. 8A has a corrugated top plate and a mating corrugated bottom plate joined by a hinge, shown on the right of FIG. 8A. The left-hand ends of the top plate and the bottom plate fit together, as in snap-fitting or latching, to hold the two plates in proximity, or in a closed position. The container is opened by separating the left-hand ends of the top plate from the bottom plate, and the planar preform is placed so as to contact the bottom plate along its surface that opposed the top plate. The top plate is returned to the closed position and the planar preform is compressed between the top plate and the bottom plate.

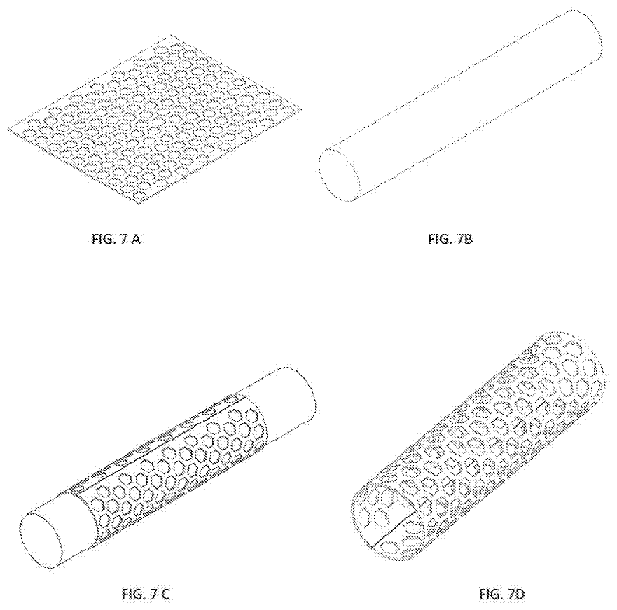

FIG. 8B shows a force-applying and/or shape-maintaining container in which a double layer corrugated degradable polymeric medical device is formed. As used herein, single and double layers refer to layers of the planar preform used in a medical device, not the number of layers of fibers laid down in printing a planar preform. The container of FIG. 8B has a corrugated top plate, a corrugated mating middle plate and a mating corrugated bottom plate, all joined by a hinge, shown on the right of FIG. 8B. The left-hand ends of the top plate and the bottom plate fit together, as in snap-fitting or latching, to hold the three plates in proximity, or in a closed position. The container is opened by separating the left-hand ends of the top plate from the bottom plate, and separating the middle layer from the bottom layer, and the planar preform is placed so as to contact the bottom plate along its surface that opposed the middle plate. For example, a planar preform in a rectangular shape as shown in FIG. 7A is placed in the container. A first end or edge of the planar preform is placed proximally to or contacting the hinge on the bottom plate. The middle plate is then placed so it contacts the top surface of the planar preform and compresses the planar preform between the mating corrugations of the bottom and middle plates. An edge or end opposite the first end of the planar preform overhangs the lefthand end of the container and is then folded over the lefthand end of the middle plate so that the uncompressed remainder portion of the planar preform contacts the top surface of the middle plate and the edge or end opposite the first end is proximal to the hinge. The top plate is returned to the closed position and the planar preform is compressed between the mating corrugations of the top plate and the middle plate, and between the middle plate and the bottom plate.

After a time period in which at least 50% of crystallization has occurred in the contained preform, which after such crystallization, a structurally stable degradable polymeric medical device is formed. The degradable polymeric medical device may be removed from the container, or it may optionally undergo post-treatments, or may remain in the container and optionally undergo post-treatments. The degradable polymeric medical device is shipped and/or stored. Such a degradable polymeric medical device may be used to in procedures to separate anatomical structures or anatomical surfaces.

1. A method of making a nasal splint, comprising,
a) 3-D printing with a degradable polymeric composition, in a continuous fiber stream, a pre-form nasal splint having a planar body;
b) contacting the pre-form nasal splint with a force-applying and/or shape-maintaining container and/or a force-applying and/or shape-maintaining component so as to shape the pre-form nasal splint into a structurally stable nasal splint.

2. The method of claim 1, wherein the degradable polymeric composition comprises at least one degradable fiber.

3. The method of claim 2, wherein the at least one degradable fiber comprises monomeric or polymeric subunits comprising L,L-lactide, D,L-lactide, glycolide, substituted glycolides, para-dioxanone, 1,5-dioxepan-2-one, trimethylene carbonate, epsilon-caprolactone, alpha-Angelica lactone, gamma-valerolactone and delta-valerolactone; glycolic acid; ethylene glycol; hydroxy-alkanoate; caprolactone; orthoesters; phosphazene; polyesters, polyether esters, hydroxybutyrate; polycarbonate, trimethylene carbonate; esteramides; anhydrides; dioxanone; alkylene alkylate; degradable urethane; etheresters; acetals; succinimides; sebacic acid, adipic acid, terephthalic acid; imino carbonates; phosphates, polyphosphonates, polyphosphazenes; poly(lactide); poly(glycolide); poly(lactide-co-glycolide); poly(lactic acid); poly(glycolic acid); poly(lactic acid-co-glycolic acid); poly(lactide)/poly(ethylene glycol) copolymers; polyglycolic acid (PGA), polylactic acid (PLA), lactic acid-glycolic acid copolymer (PLGA), polyhydroxyalkanoates (PHA), polyhydroxybutyrate-valerate (PHBV), polyvinyl alcohol (PVA), polyethylene terephthalate (PET), polyglycolide-lactide, polycaprolactone (PCL), lactic acid-ε-caprolactone copolymer (PLCL), polydioxanone (PDO), polytrimethylene carbonate (PTMC), poly(amino acid), polydioxanone, polyoxalate, a polyanhydride, a poly(phosphoester), polyorthoester and copolymers thereof, poly hyaluronic acid; poly(glycolide)/poly(ethylene glycol) copolymers; polyether-ester polymers, poly(para-dioxanone).a polyhydroxy-alkanoate, poly(lactide-co-glycolide)/poly(ethylene glycol) copolymer; poly(lactic acid)/poly(ethylene glycol) copolymer; poly(glycolic acid)/poly(ethylene glycol) copolymer; poly(lactic acid-co-glycolic acid)/poly(ethylene glycol) copolymer; poly(caprolactone); poly(caprolactone)/poly(ethylene glycol) copolymer; poly(orthoester); poly(phosphazene); poly(hydroxybutyrate) or copolymer including a poly(hydroxybutyrate); poly(lactide-co-caprolactone); polycarbonate, poly(trimethylene carbonate); polyesteramide; polyanhydride; poly(dioxanone); poly(alkylene alkylate); copolymer of polyethylene glycol and a polyorthoester; degradable polyurethane; poly(amino acid); polyetherester; polyacetal; polycyanoacrylate; poly(oxyethylene)/poly(oxypropylene) copolymer, polysuccinimide; a polyanhydride poly(sebacic acid), poly(adipic acid), poly(terephthalic) acid; polyamide; poly(imino carbonate) polyamino acid; phosphorus-based polymer; polyphosphate, polyphosphonate, or polyphosphazene; or combinations thereof.

4. The method of claim 2, wherein the at least one degradable fiber comprises monomeric or polymeric subunits comprising glycolide, trimethyl carbonate, and caprolactone monomeric subunits.

5. The method of claim 4, wherein the at least one degradable fiber comprises from about 50% to about 60% glycolide subunits, from about 20% to about 30 trimethyl carbonate subunits, and from about 10% to about 30% caprolactone subunits, of the total number of subunits present within the copolymer.

6. The method of claim 1, wherein the pre-form nasal splint is a planar geometrical or non-geometrical shape.

7. The method of claim 6, wherein the planar shape is a circle, a star, a triangle, a square, a parallelogram, an octagon, or a rhomboid or a random undefined shape.

8. The method of claim 1, wherein confining the pre-form nasal splint comprises placing the pre-form nasal splint in a force-applying and/or shape-maintaining mold.

9. The method of claim 1, wherein confining the pre-form nasal splint comprises placing the pre-form nasal splint in a force-applying and/or shape-maintaining container.

10. The method of claim 8, further comprising contacting the pre-form nasal splint with one or more force-applying and/or shape-maintaining components.

11. The method of claim 8, wherein the force-applying and/or shape-maintaining mold or container forms a tubular shape in a portion of the pre-form nasal splint.

12. The method of claim 1, wherein the pre-form nasal splint is confined in a force-applying and/or shape-maintaining mold or container for at least 24 hours post-printing.

13. The method of claim 1, wherein the nasal splint is not sterile.

14. The method of claim 1, wherein the nasal splint is sterilized.

15. The method of claim 1, wherein the nasal splint, confined within the force-applying and/or shape-maintaining mold or container, is exposed to ionizing radiation to enhance degradation of at least a portion of the polymeric material of the nasal splint.

16. The method of claim 1, further comprising shipping the nasal splint contained within the force-applying and/or shape-maintaining mold or container.
